(19) United States
(12) Patent Application Publication
(10) Pub. No.: US 2005/0065530 A1
(43) Pub. Date: Mar. 24, 2005

(54) Internal saw for osteotomy of tubular bones

(76) Inventors: Roman Stauch, Igersheim (DE); Jurgen Klein, Erneen (DE)

Correspondence Address: BACHMAN & LAPOINTE, P.C., 900 CHAPEL STREET, SUITE 1201, NEW HAVEN, CT, 06510, US

(21) Appl. No.: 10/496,184

(22) PCT Filed: Aug. 13, 2002
(86) PCT No.: PCT/EP02/09048

(30) Foreign Application Priority Data
Nov. 14, 2001 (DE) 101 55 739.6
Dec. 7, 2001 (DE) 101 60 232.4

(51) Int. Cl. A61B 17/32

(52) U.S. Cl. 606/82

(57) ABSTRACT

An internal saw for osteotomy of tubular bones, comprising a base body, provided with a saw blade, which is moved radially out of the base body, whereby the base body, in particular the saw blade may be driven to oscillate radially.

### BACKGROUND OF THE INVENTION

The present invention relates to an internal saw for osteotomy of tubular bones, comprising a base body provided with a saw blade which can be moved radially out of the base body.

An internal saw of this kind for tubular bones is known from German utility model G 92 17 188.5, for example. With an internal saw of this kind, a tubular bone can be cut through with a saw and a saw unit. The saw emerges radially from the base body and cuts through the bone as the base body is turned. Particularly in surgery, bones have to be cut through radially, for example, if bone or bone fractures do not fuse correctly or if it is necessary to correct malformed or curved bones or extremities which are too long or too short. Internal saws of this kind are usually guided into the tubular bone from the end in order to cut through the bone from the inside outward. Particularly in distraction of bones, a medullary nail, a distraction device or the like is inserted into the axial bore or into an axial access after the tubular bone has been cut through.

A disadvantage of the conventional known internal saw is that it is awkward to operate and difficult to insert and control. In particular, when the above-described internal saws are used, the periosteum and surrounding tissue are often damaged when cutting through the tubular bone, which is undesirable. This slows down the course of healing.

U.S. Pat. No. 5,591,107 describes an oscillating internal saw for osteotomy of tubular bones, where a manually operated lever is used to influence an advance movement of the radial saw, which emerges radially from a base body. In addition, this manually operated lever can be used to effect a rotation movement.

WO 01/34040 A1 discloses an internal saw for cutting through tubular bones, where two tools mounted like scissors are moved apart from one another by means of centrifugal force during rotation, these tools bearing internally on the tubular bone and thus cutting through the tubular bone upon rotation. A disadvantage here is that this device can only be used to cut through bones of large internal diameter.

It is an object of the present invention to make available an internal saw of the type mentioned at the outset which eliminates the above-mentioned disadvantages and with which it is also possible to exert an influence on different bone cross sections, so that these are cut though uniformly. Such an internal saw should also be easy to operate and inexpensive to produce.

### SUMMARY OF THE INVENTION

The foregoing object is achieved by the present invention wherein a saw blade can be very easily exchanged and advanced. For example, an operating element can be assigned to a jacket surface of the base body, which operating element can be operated by means of a finger of a human hand and can be axially displaced. The saw blade, which directly adjoins the operating element, can be pushed radially out from one end of the base body in a graduated manner, using the fingers of a human hand, in order to advance or orient the saw blade. At the same time, the base body can be turned radially about a center axis in order to perform a sawing operation. It is important, however, that, during the radial turning of the base body, the latter, and thus also the saw blade, performs an oscillating movement by way of a drive device. This oscillating movement improves the cutting action and at the same time ensures that saw blades can be used which in principle do not damage the periosteum or the bone itself. The saw blade obtains its cutting and separating action through the oscillating movement alone, but only for hard objects, such as for example the tubular bone. When cutting through the tubular bone, by radial advance of the saw blade and simultaneous oscillation, the saw blade abuts the periosteum but does not damage it in the process.

A further advantage of the present invention is that the advance of the saw blade can be graduated manually, with or without locking, so that the surgeon can read off how deep he has cut through the tubular bone. This advance movement can also be prestressed by means of a spring element which engages on the operating element or directly on the saw blade. The invention is not intended to be limited to this.

In a further illustrative embodiment of the present invention, it has proven particularly advantageous to operate the saw blade, in particular the operating element, by means of an actuator, if appropriate of a control means attached to it, a force sensor being provided between actuator and saw blade or between actuator and operating device. The control means is able to provide the actuator, for example, with an input corresponding exactly to CAD/CT cross-sectional data of the bone, which then correspondingly controls the depth of advance or a return movement of the saw blade depending on the radial angular position with respect to the bone.

By this means, a bone which does not have a circular cross section can also be sawn through uniformly since the advance of the saw blade takes place according to the cutting data and the cross-sectional profile of the bone. A computer-aided control means can provide for an advance depth of the saw blade according to the cutting data or cross-sectional profile of the tubular bone. This is also intended to lie within the scope of the present invention.

It has also proven particularly advantageous that a drive device at the same time transmits, via an oscillation gear, a radial oscillating movement to the base body, in particular to the saw blade, and at the same time can perform a radial complete rotation in a selectable direction for radially cutting through the bone.

### BRIEF DESCRIPTION OF THE DRAWINGS

Further advantages, features and details of the invention will become clear from the following description of preferred illustrative embodiments and from the drawing, in which:

FIG. 1a shows a diagrammatic partial longitudinal section through an internal saw for osteotomy of tubular bones;

FIG. 1b shows a diagrammatic partial longitudinal section through a further illustrative embodiment of an internal saw according to FIG. 1a;

FIG. 1c shows a diagrammatic partial longitudinal section through a further illustrative embodiment of an internal saw according to FIGS. 1a and 1b;

FIG. 2 shows a diagrammatic partial longitudinal section through an internal saw in a possible position of use;

FIG. 3 shows a diagrammatic partial longitudinal section through the internal saw with associated drive device according to FIG. 1a;

FIG. 4 shows a diagrammatic side view of the internal saw, in particular of a base body in a position of use according to FIG. 1a;

FIG. 5 shows a diagrammatic side view of part of the saw blade, particularly in the end area;

FIG. 6 shows a diagrammatic partial longitudinal section through a tubular bone with inserted internal saw as a further illustrative embodiment.

### DETAILED DESCRIPTION

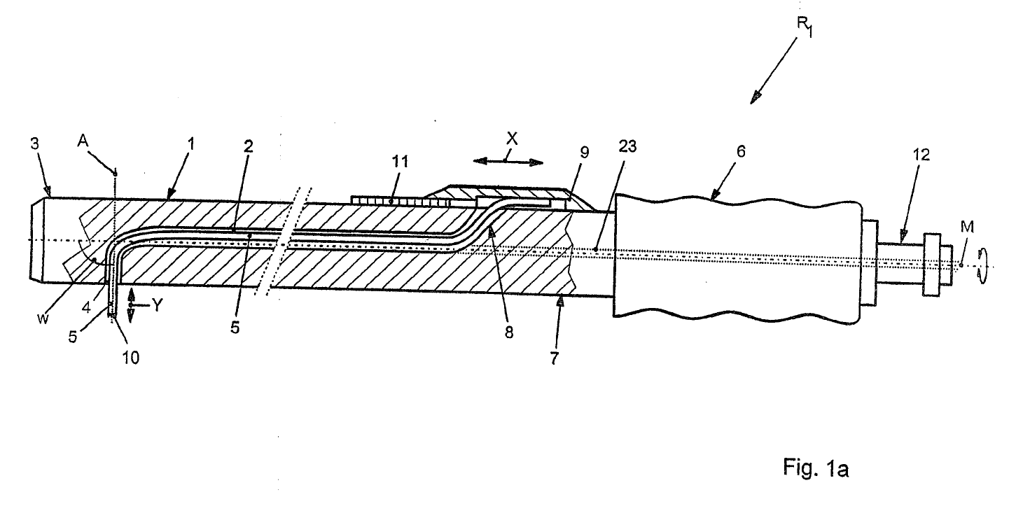

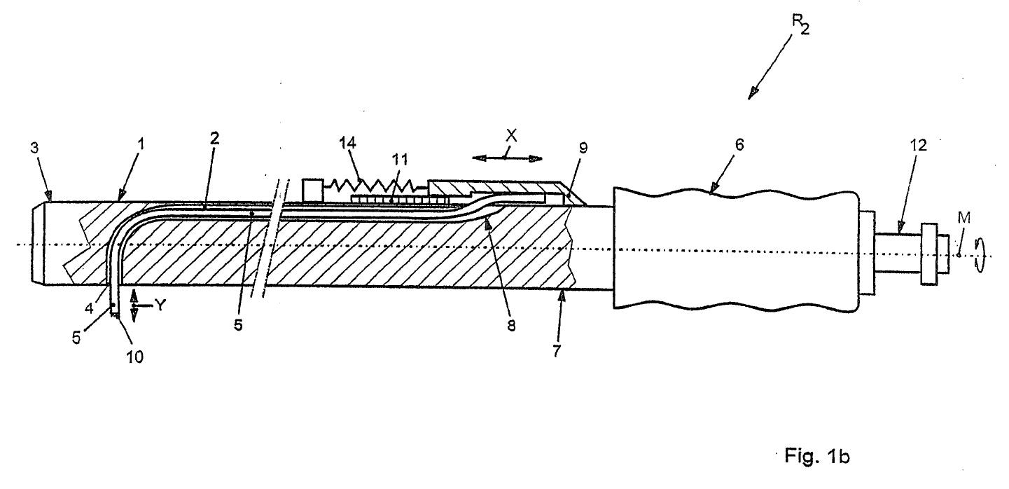

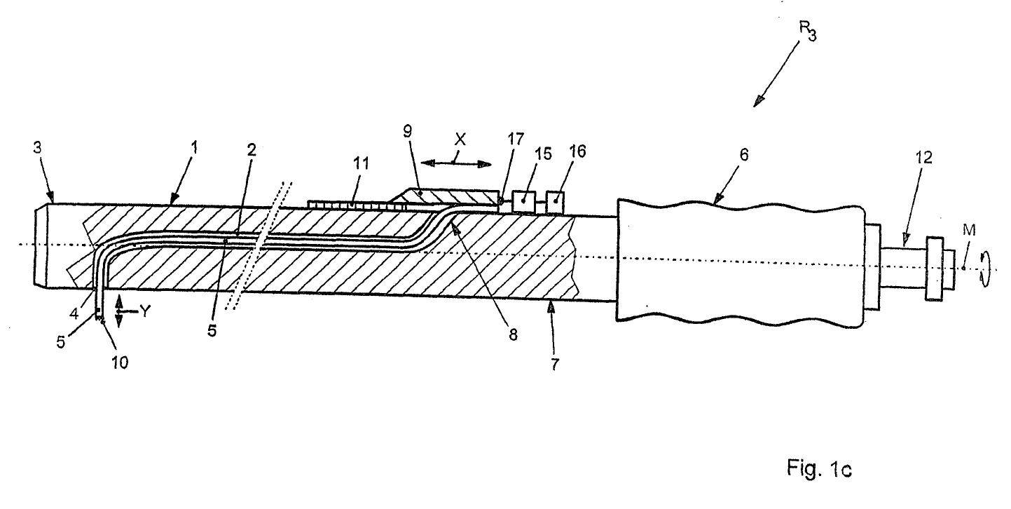

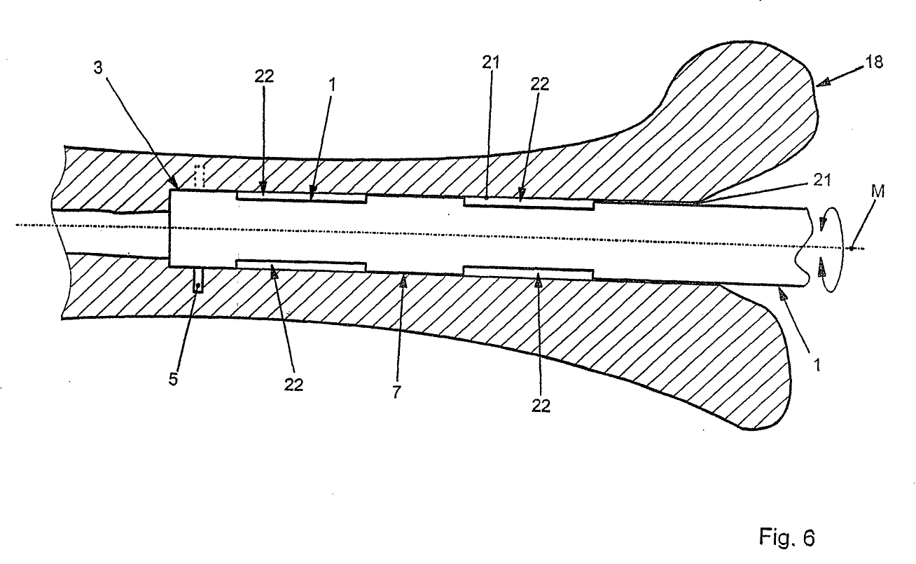

According to FIG. 1a, an internal saw R₁ according to the invention has a base body 1 in which a saw blade 5, preferably of rectangular cross section, is guided axially, preferably centrally, in a guide 2, and emerges radially from an outlet opening 4 in a head area 3 of the base body 1. At the other end of the head area 3, near to a grip element 6, an insertion shaft 8 serving for insertion of the saw blade 5 is formed on an outer jacket surface 7, preferably opposite to the outlet opening 4, in the base body 1. The saw blade 5 can be connected permanently or releasably to an operating element 9 or can be permanently connected, and the saw blade 5 can be moved radially out of the head area 3 of the base body 1 by movement of the operating element 9 along the double arrow direction X shown. At the front end, the saw blade 5 is provided, as can be seen in particular in FIG. 5, with a plurality of teeth 10 which are arranged in an arc shape, for example, in the front end area. The saw blade 5 is preferably curved at the front end.

By movement of the operating element 9 in the double arrow direction X shown, the end area of the saw blade 5 is moved radially into or out of the base body 1 in the double arrow direction Y shown.

As can further be seen from FIG. 1a, the operating element 9 can be operated, for example, by a finger of a human hand and can be moved along a graduation 11 which corresponds to a depth of advance of the saw blade 5. In this way, a depth of advance can be exactly set and read off from the outside.

It is further of advantage that the saw blade 5, for example in the case of single use, is simply withdrawn from the insertion shaft 8 and can be very easily exchanged. If appropriate, the operating element 9, with fixed saw blade 5, can be inserted again into the base body 1 through the insertion shaft 8 for renewed use. The invention is not limited to this.

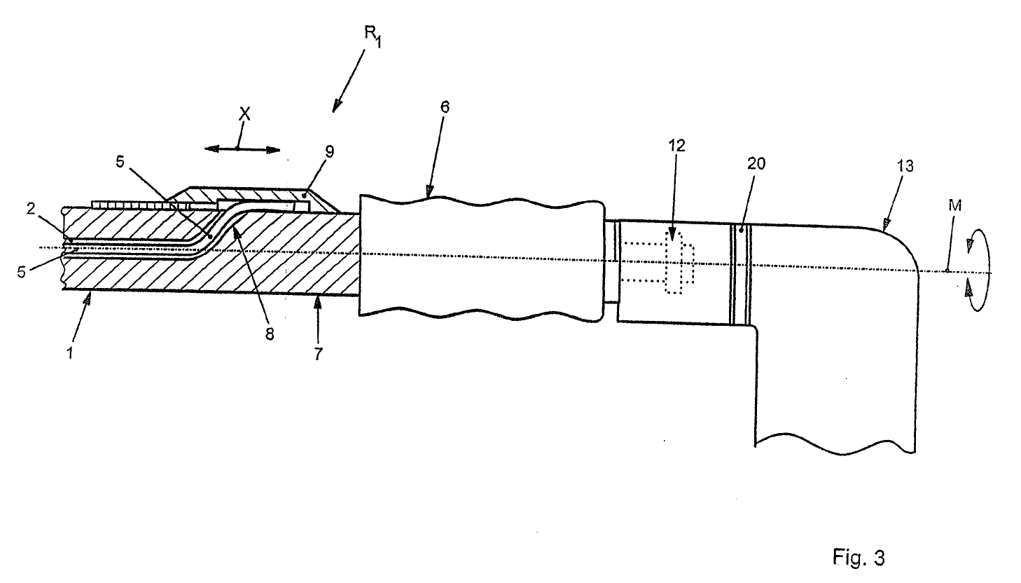

It is important, however, that at one end of the base body 1 the grip eIement 6 is radically and rotationally uncoupled with respect to the base body 1 so that the grip element 6 can, for example, be held by fingers of a human hand, so that the base body 1 can be driven manually and/or automatically about a center axis M. At the other end of the grip element 6, an attachment 12 is provided, preferably as an AO attachment, for receiving a drive device 13, as is shown, for example, in a simplified form in FIG. 3.

The drive device 13 can also simply be a manual grip with which the base body 1 can be moved in a rotation movement about the center axis M, the grip element 6 being uncoupled from the rotation movement about the center axis M.

In the area of the outlet opening 4, the saw blade 5 lies on an axis A which, in the present case, encloses an angle W, preferably of 90°, between the center axis M.

The outlet opening 4 is oriented in the base body 1 at an angle W of 90° with respect to the center axis M. It has proven advantageous to provide the outlet opening 4 in the base body 1 at a smaller angle of between 40° and 90°, for example. In this way, the bones can be cut through in inclined cutting surfaces, which results in an enlargement of the cutting surfaces. Thus, in the case of quite large cutting surfaces, it is also of advantage here that more rapid healing is provided and, in particular, greater stability upon fusion of a large cutting surface.

The outlet opening 4 can be oriented at an angle W of, for example, 40° to 140° between axis A and center axis M. Changing the angle W even during operation, for example by means of a device not shown here, is also intended to lie within the scope of the present invention.

It has also proven advantageous to have a flushing channel 23 which opens into the insertion shaft 8 and which can serve for flushing and/or aspiration. The base body 1 can also be cooled at the interface via the flushing channel 23.

It is also conceivable for the operating element 9 and also the graduation 11 to be provided all round the base body 1, so that the operating element 9 can be gripped in any desired position and the graduation 11 can be read off in any desired position. In this case, the graduation 11 is arranged all the way round the jacket surface 7 and can, if appropriate, include a plurality of different graduations 11. The operating element 9 would then also be designed as an annular sleeve or the like which engages completely or at least partially around the jacket surface 7 of the base body 1.

In a further illustrative embodiment of the present invention according to FIG. 1b, a further internal saw R₂ is shown which corresponds substantially to the type described above.

The difference is that the guide 2 forms a greater radius for leading the saw blade 5 from the outlet opening 4 to the insertion shaft 8. If appropriate, a groove can be formed in the base body 1 in the area of the insertion shaft 8 in the jacket surface 7, in order to guide or accommodate the saw blade therein.

In the manner described above, the saw blade 5 can be moved to and fro via the operating element 9 along the graduation 11 in the direction shown by the double arrow X, in order to move the saw blade 5, in particular its saw teeth 10, out of the outlet opening 4.

It is also conceivable, here, to move the operating element 9 with locking relative to the graduation 11.

The scope of the present invention is also intended to include the operating element 9 being adjoined by a spring element 14, which is designed as a compression and/or tension spring, in order to prestress the operating element 9 toward the head area 3. It is also conceivable to alter the spring constantly and to vary the prestressing force of the spring element 14.

In a further illustrative embodiment of the present invention according to FIG. 1c, an internal saw R₃ is shown which corresponds substantially to the internal saw R₁ according to FIG. 1. The difference is that the saw blade 5 and/or the operating element 9 can be moved to and fro by means of an actuator 15 in the direction shown by the double arrow X. The actuator 15 is controlled via a control means 16.

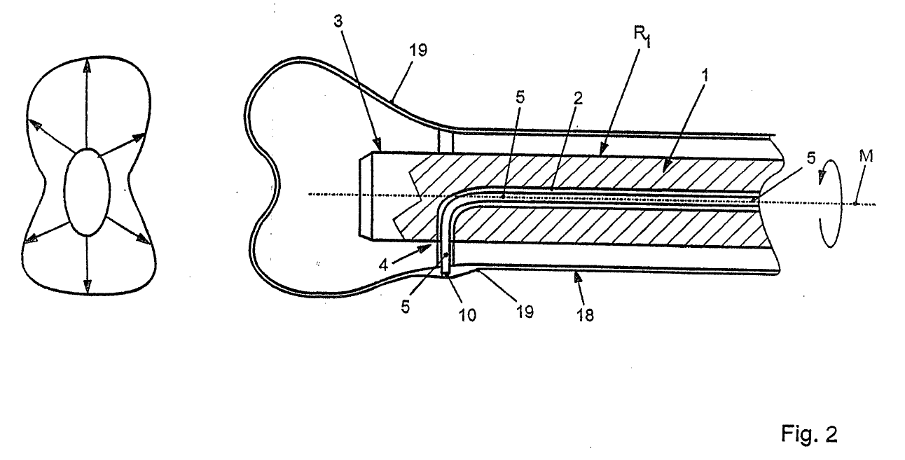

A force sensor 17 can be provided between actuator 15 and operating device 9 and/or between actuator 15 and saw blade 5, in order to exactly measure a permanent force, in particular an advance force. This is important, for example, if the saw teeth 10 cut completely through a tubular bone 18, see FIG. 2, so that periosteum 19 lying thereon is not damaged.

It has also proven advantageous that, via the control means 16, the actuator 15 is able to adapt an advance movement of the saw blade, as it is shown in particular in FIG. 2, according to the cross-sectional profile of the tubular bone. A corresponding cross section can be determined by ultrasound, for example, and different cutting depths for cutting through the tubular bone 18 can accordingly be input via the control means 16, so that, depending on the position of the internal saw R₃, and depending on the position of the internal saw R₃ with respect to the bone, a different depth can be set in accordance with the cross-sectional shape of the tubular bone 18 to be cut through. These data can, for example, also be delivered by wireless transmission to the control means 16 and thus to the actuator 15.

In the illustrative embodiment according to FIG. 2, the internal saw R₁ is shown in a position of use in which, by radial turning of the base body 1 about the center axis M and simultaneous advance of the saw blade 5, the tubular bone 18 is cut through.

An important aspect of the present invention is that the depth of advance of the saw blade 5, in particular in the area of the outlet opening 4, is different depending on the position and cross section of the bone, since the tubular bone 18 is thicker or thinner at different angle areas and has different radii with respect to the center axis. In order to compensate for this, a depth of advance can be permanently varied on rotation of the internal saw R₁. Preferably, the advance of the saw blade 5 is controlled automatically in accordance with the cross section of the tubular bone 18.

An important aspect of the present invention is, however, as is shown in particular in FIG. 3, that the drive device 13 can be connected to the base body 1, particularly in the area of the attachment 12, and an oscillation gear 20 is provided which sets the base body 1 into a radial oscillating vibration movement. At the same time, the drive device 13 can be turned manually and radially about the center axis M in relation to the tubular bone 18 (not shown here) and in relation to the grip element 6, so that the saw blade 5 cuts through the tubular bone 18 by means of the radial turning. By means of the oscillating movement, i.e. the radial movement to and fro of the base body 1, and thus also of the saw blade 5, it is possible to achieve optimized cutting of the tubular bone 18, without the periosteum 19 being damaged as the saw blade 5 passes through the tubular bone 18. An oscillating movement of the saw blade 5 does not by itself damage the periosteum 19. At the same time, a rotation movement about the center axis M can be effected manually and/or automatically via the drive device 13, this rotation movement being added to or superposed on the oscillating movement, so that the tubular bone 18 is cut through over its complete cross section. If the saw hits the periosteum 19, the advance is automatically halted and the saw blade 5 drawn back.

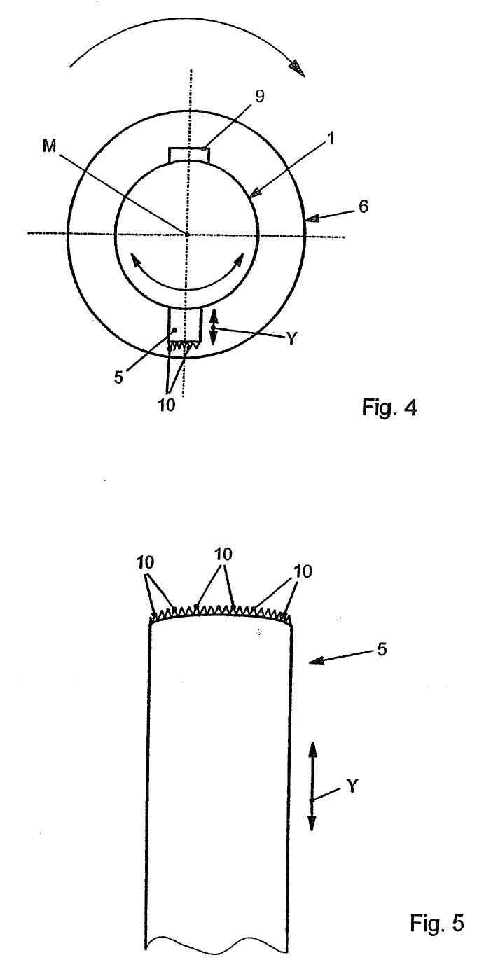

FIG. 4 shows how the saw blade 5 can be moved out of the base body 1 in the Y direction shown, and the base body 1 performs an oscillating movement about the center axis M and can be turned completely about the center axis M in an associated or superposed movement, and the person using the operating element 9 identifies in which radial position the saw blade 5 is located in the tubular bone 18. This is also intended to lie within the scope of the present invention.

Another important aspect of the present invention is that the base body 1, as is shown in particular in FIG. 6, is inserted into a bore 21 of a tubular bone 18. The base body 1 will be inserted with an exact fit into the bore 21 of the tubular bone 18. Since the base body 1 is turned radially about the center axis M as the saw blade 5 cuts through the bone, it has proven particularly advantageous to provide a plurality of steps 22 in the jacket surface 7 of the base body 1, their diameter being smaller than the remaining part, for example than a head area 3 of the base body 1. Thus, the base body 1 bears against the tubular bone 18 only at a few locations, so that the tubular bone 18 is cut through by means of radial turning, with extremely low development of heat in the tubular bone 18, which fact contributes to the preservation of the tissue and to growth. In this way, vessels of the bone, the periosteum etc., are treated much more gently and exposed to less stress. A rotation movement of the base body 1 has reduced friction and is thus easier.

1-23: (cancelled):

24: An internal saw for osteotomy of tubular bones, comprising a base body, a saw blade, and means for moving the saw blade in and out of the base body radIally of the axis (M), wherein the saw blade oscillates radially.

25: An internal saw for osteotomy of tubular bones, comprising a base body having an elongated axis (M), a saw blade, and means for moving the saw blade in and out radially of the axis (M), wherein the means for moving the saw blade in and out of the base body comprises at least one operating element which is manually actuated linearly.

26: An internal saw for osteotomy of tubular bones, comprising a base body having an elongated axis (M), a saw blade, and means for moving the saw blade in and out of the base body, radially of the axis (M), wherein advance movement of the saw blade is controlled as a function of cross section and radial position of the saw blade with respect to a tubular bone.

27: An internal saw for osteotomy of tubular bones, comprising a base body having an elongated axis (M), a saw blade, and means for moving the saw blade in and out of the base body, radially of the axis (M), wherein a plurality of steps are provided in a surface of the base body.

28: The internal saw as claimed in at least one of claims 24 through 27, wherein the base body includes a grip element is rotationally uncoupled radially with respect to the base body.

29: The internal saw as claimed in claim 28, wherein the base body has an attachment for the means for moving.

30: The internal saw as claimed in claim 29, wherein the means for moving comprises a drive device for transmitting oscillating and/or radial movement of rotation to the attachment, to the base body, and thus to the saw blade.

31: The internal saw as claimed in claim 25, wherein the saw blade is exchangeable and removable from an insertion shaft with or without operating element.

32: The internal saw as claimed in claim 25, wherein the base body is provided with a graduation in the area of the operating element, and, by linear movement of the operating element along the graduation, the saw blade is radially moved out of the base body by a corresponding path of displacement.

33: The internal saw as claimed in claim 32, wherein the operating element is moved with possible locking along the graduation, wherein the operating element and the graduation comprises as a slide element around a surface of the base body.

34: The internal saw as claimed in claim 30, wherein the base body and the saw blade are radial oscillated by the drive device, and radial rotated about axis (M) by the drive device.

35: The internal saw as claimed in claim 30, wherein the drive device is manually turned in a radial direction in order to cut through a tubular bone with simultaneous oscillating movement of the base body.

36: The internal saw as claimed in claim 30, wherein the drive device is connected to the attachment and drives the base body radially in oscillating motion.

37: The internal saw as claimed in claim 28, wherein at least one step is formed between a head area of the base body and the grip element, wherein each of the steps has a diameter which is smaller than a diameter of the head area.

38: The internal saw as claimed in claim 26, wherein at least one actuator is attached to the end of one of the saw blade and the operating element.

39: The internal saw as claimed in claim 38, wherein at least one force sensor is provided between the actuator and one of the saw blade and operating element.

40: The internal saw as claimed in claim 39, wherein the actuator is adjoined by a control means which controls an advance movement and return movement of the saw blade for applying a force of the saw blade onto a tubular bone to be cut.

41: The internal saw as claimed in claim 40, wherein different sawing depths of a tubular bone are input by the control means to the actuator for adjusting sawing depth of the saw blade on the tubular bone.

42: The internal saw as claimed in claim 40, wherein the advance and return movement of the saw blade takes place according to defined cross-sectional data of the tubular bone to be cut.

43: The internal saw as claimed in at least one of claims 24 to 27, wherein the saw blade has a curved configuration at a front end thereof.

44: The internal saw as claimed in a least one of claims 24 to 27, wherein the base body has a radial an outlet opening which lies on an axis (A) which forms an angle (W) of 40° to 140° relative to axis (M).

45: The internal saw as claimed in claim 44, wherein the angle (W) of the outlet opening between axis (A) and center axis (M) is smaller than or greater than 90° to provide an inclined cutting surface.

46: The internal saw as claimed in at least one of claims 24 to 27, wherein at least one flushing channel adjoins an insertion shaft for the saw blade.
